(19) United States
(12) Patent Application Publication
(10) Pub. No.: US 2026/0262947 A1
(43) Pub. Date: Sep. 10, 2026

(54) LASER OUTPUT CALIBRATION SYSTEM FOR AN OPTOACOUSTIC PROBE

(71) Applicant: Seno Medical Instruments, Inc., San Antonio, TX (US)

(72) Inventors: Tam Do, San Antonio, TX (US); Bryan Clingman, San Antonio, TX (US); Allison Bertrand, San Antonio, TX (US); Scott Miller, San Antonio, TX (US); Yunus Abdussalam, San Antonio, TX (US)

(73) Assignee: Seno Medical Instruments, Inc., San Antonio, TX (US)

(21) Appl. No.: 19/447,723

(22) Filed: Jan. 13, 2026

Related U.S. Application Data
(60) Provisional application No. 63/744,945, filed on Jan. 14, 2025.

(51) Int. Cl. A61B 5/00

(52) U.S. Cl. CPC A61B 5/0095; A61B 5/742

(57) ABSTRACT

An optoacoustic probe system includes an optoacoustic probe with a light source configured to generate light, a first energy output sensor configured to detect signals from the light a second energy output sensor configured to detect the signals, and a microcontroller. A processor of the microcontroller can be configured to receive the signal and vary the at least one light source based on the signals detected, determine a first energy output based on the signals, vary an input to the at least one light source based on the first energy output, determine a second energy output based on the signals detected by the second energy output sensor, compare the first energy output to the second energy output, and provide an alert or turning off the at least one light source based on comparing the first energy output to the second energy output.

### CROSS REFERENCE TO RELATED APPLICATIONS

This application claims benefit to U.S. Provisional Application No. 63/744,945, filed on Jan. 14, 2025, titled “LASER OUTPUT CALIBRATION SYSTEM FOR AN OPTOACOUSTIC PROBE”, the complete subject matter of which is expressly incorporated herein by reference in its entirety.

### TECHNICAL FIELD

The present invention relates in general to the field of medical imaging, and in particular to a system relating to optoacoustic imaging.

### BACKGROUND

Optoacoustic imaging systems visualize thin tissue slices noninvasively through skin at a tissue site. A tissue site may contain a variety of tissue structures that may include, for example, tumors, blood vessels, tissue layers, and components of blood. In optoacoustic imaging systems, light is used to deliver optical energy to a planer slice of the tissue site, which as a result of optical absorption with the tissue structures, produces acoustic waves. An image spatially representing the tissue site can be generated by performing image reconstruction on acoustic signals that return to an ultrasound transducer array. Because biological tissue scatters impinging optical energy in many directions the optical energy can be absorbed by tissue structures outside of a targeted region, which can generate acoustic return signals that interferes with the imaging of tissue structures within the targeted region.

A laser light source typically provides the optical energy required to generate the acoustic waves. During such operation, great care must be taken to ensure the emitted optical energy does not harm a patient and clinician, including the eyes of the patient or clinician.

In order to prevent such harm, safety glasses are often worn by the clinician during such procedures. Additionally, probe holders are utilized that absorb or prevent the emission of the radiation, so that if a clinician forgets to turn off the probe, the radiation cannot harm any individuals in the environment. In other examples, the probe does not emit optical energy unless a foot actuator is utilized. Still, when the probe is removed from the holder, or when a clinical drops the probe while utilizing the foot actuator, potentially harmful optical energy may be emitted into the environment, increasing the chances of harm.

Standards have been developed in relation to laser light sources. For example, there are standards that require that the lasers keep an output energy within an 80%-120% range of a target setpoint. Keeping the output energy in this range ensures that proper functionality for imaging is achieved while also reducing harmful radiation emissions into the environment. One way to keep the output energy of the laser in the 80%-120% range is to provide a pyroelectric sensor that is used to determine the amount of energy that is coming out of the probe, while at the same time collecting reference energy readings using a photodiode from the output light path. Such readings can be provided to software that fits the reference energy readings to a calibrated energy sensor readings to perform calibration initially, and then a feedback loop with curve fitting to continuously adjust the laser to stay within the 80%-120% range.

In applications such as medical devices and safety critical systems, it is required for systems to be fail-safe and redundant. This can be accomplished by monitoring additional energy sensors, and control signals to the laser system.

Still, problematically, hardware inaccuracies such as a malfunctioning or inaccurate photodiode sensor and motor positioning, can result in errors in readings. Additionally slow or sudden component failures of coatings on optical components can occur which can change the output energy relationship. Such inaccurate readings when used within a feedback loop can lead the laser to fall outside of the 80%-120% range. As a result, accurate imaging can be compromised, or the laser can emit an energy output into an environment that is greater than the 120% maximum desired. Consequently, a system can be provided that determines a confidence level related to the accuracy of the energy output levels being detected. Still, such confidence level can be marred by the underlying data, be expensive to implement, and not always easy to use.

Additionally, laser components can wear out or change alignment over time. A sudden change in laser output performance can result in costly downtown for a customer. Being able to monitor and anticipate laser failures is important to ensure that system downtime is minimized.

A need therefore exists for a more effective design for a laser output calibration system for an OA probe to determine the reliability energy output of a laser at any given time, and the health of the laser system.

### BRIEF SUMMARY

New and useful systems, apparatuses, and methods for providing optoacoustic imaging are set forth in the appended claims. Illustrative embodiments are also provided to enable a person skilled in the art to make use of the claimed subject matter.

Objectives, advantages, and a preferred mode of making and using the claimed subject matter may be understood best by reference to the accompanying drawings in conjunction with the following detailed description of illustrative embodiments.

In accordance with embodiments herein, an optoacoustic probe system for optoacoustic imaging of a volume is provided. The optoacoustic probe system can include an optoacoustic probe having a distal end operable to contact the volume and a proximal end. The optoacoustic probe can include at least one laser light source outside a probe housing configured to generate light that is transmitted along a light path to generate optoacoustic return signals when the light reacts with the volume, a primary sensor configured to detect signals from the light generated by the at least one laser light source, and a secondary sensor configured to detect the signals from the light generated by the at least one laser light source. The microcontroller may include one or more processors, and a memory coupled to the one or more processors, wherein the memory stores program instructions. The program instructions are executable by the one or more processors to detect signals from the light with the primary sensor, determine a first energy output based on the signals detected by the primary sensor, detect the signals with the secondary sensor, determine a second energy output based on the signals detected by the secondary sensor, compare the first energy output to the second energy output, and provide an alert or turning off the at least one laser light source based on comparing the first energy output to the second energy output.

Optionally, the one or more processors can be further configured to determine whether the first energy output is within a range of the second energy output based on the comparison and provide the alert or turning off the at least one laser light source in response to a determination that a comparison between the first energy output to the second energy output falls outside of the range. In one aspect, the one or more processors can be further configured to vary an input to the at least one laser light source by determining whether the first energy output is within an expected power output range related to the at least one laser light source and varying the input in response to the first energy output falling outside of the expected power output range. In another aspect, the input can be a voltage input to the at least one light source. In one example, the at least one light source can be a Alexandrite laser. In another example, the at least one laser light source can be disposed within a chassis.

Optionally, the primary sensor can be disposed within the chassis adjacent the at least one laser light source. In one aspect, the secondary sensor can be disposed within the chassis adjacent the at least one laser light source. In another aspect, the primary sensor can be at least one of a photodiode sensor or a pyroelectric sensor. In one example, the secondary sensor can be at least one of a photodiode sensor or a pyroelectric sensor. In another aspect, the one or more processors can be configured to provide the alert and to provide the alert includes displaying on a display a message. In yet another example, the display can be configured to display an optoacoustic image.

In accordance with embodiments herein an optoacoustic probe system for optoacoustic imaging of volume is provided that includes an optoacoustic probe having a distal end operable to contact the volume and a proximal end, the optoacoustic probe and a chassis having at least one laser light source configured to generate light that is transmitted along a light path between the chassis and optoacoustic probe to generate optoacoustic return signals when the light reacts with the volume. The optoacoustic probe system can also include a display disposed on the chassis and configured to display an optoacoustic image based on the optoacoustic return signals, a primary sensor configured to detect signals from the light generated by the at least one laser light source, and a secondary sensor configured to detect the signals from the light generated by the at least one laser light source. The optoacoustic probe system can also include a microcontroller including one or more processors, and a memory coupled to the one or more processors, wherein the memory stores program instructions. The program instructions can be executable by the one or more processors to detected the signals with the primary output sensor, determine a first energy output based on the signals detected by the first energy output sensor, detect the signals with the secondary output sensor, determine a second energy output based on the signals detected by the secondary sensor, compare the first energy output to the second energy output, and display an alert on the display based on comparing the first energy output to the second energy output.

Optionally, the one or more processors can be further configured to determine whether comparing the first energy output and the second energy output is within a first range and display the alert when the comparison is within the first range. In one aspect, the one or more processors can be further configured to determine whether when comparing the first energy output and the second energy output is within a second range that is greater than the first range; and turn off the at least one laser light source when the comparison between the first energy output and the second energy output is outside the second range. In another aspect, the one or more processors can be further configured to vary an input to the at least one laser light source by determining whether the first energy output is within an expected power output range related to the at least one laser light source and varying the input in response to the first energy output falling outside of the expected power output range.

Optionally, the input can be a voltage input to the at least one laser light source. In one aspect, the at least one laser light source can be an Alexandrite laser. In another aspect, the primary sensor may be disposed within the chassis adjacent the at least one laser light source. In one example, the secondary sensor can be disposed within the chassis adjacent the at least one laser light source. In another example, the primary sensor can be at least one of a photodiode sensor or a pyroelectric sensor. In yet another example, the secondary sensor can be at least one of a photodiode sensor or a pyroelectric sensor.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a schematic block diagram illustrating an embodiment of an optoacoustic system that may be used as a platform for the methods and devices disclosed herein.

FIG. 2 shows a partial top plan view of a laser light system that may be used in connection with the methods and other devices disclosed herein.

FIG. 3 shows a schematic of laser light produced by the laser light system of FIG. 2 in connection with the methods and other devices disclosed herein.

FIG. 4A shows a plot of sample readings from a primary sensor in connection with the methods and other devices disclosed herein.

FIG. 4B shows a graph of output energy detected over time in connection with methods and other devices disclosed herein.

FIG. 5 shows a schematic block diagram of a control system for devices disclosed herein.

FIG. 6 shows a block flow diagram of a method of controlling light sources for the devices disclosed herein.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The systems and methods are described below with reference to, among other things, block diagrams, operational illustrations and algorithms of methods and devices to provide optoacoustic imaging with out-of-plane artifact suppression. It is understood that each block of the block diagrams, operational illustrations and algorithms and combinations of blocks in the block diagrams, operational illustrations and algorithms, can be implemented by means of analog or digital hardware and computer program instructions.

These computer program instructions can be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams, operational block or blocks and or algorithms.

In some cases, frequency domain-based algorithms require zero or symmetric padding for performance. This padding is not essential to describe the embodiment of the algorithm, so it is sometimes omitted from the description of the processing steps. In some cases, where padded is disclosed in the steps, the algorithm may still be carried out without the padding. In some cases, padding is essential, however, and cannot be removed without corrupting the data.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Reference will now be made in more detail to various embodiments of the present invention, examples of which are illustrated in the accompanying figures. As will be apparent to one of skill in the art, the data structures and processing steps described herein may be implemented in a variety of other ways without departing from the spirit of the disclosure and scope of the invention herein and should not be construed as being limited to the embodiments set forth herein. Rather, these embodiments are provided so that this disclosure will be thorough and complete, and will fully convey the concept of the disclosure to those skilled in the art.

Embodiments herein may be implemented in connection with one or more of the systems and methods described in one or more of the following patents, publications and/or published applications, all of which are expressly incorporated herein by reference in their entireties:
U.S. Pat. No. 7,999,161, titled “Laser-Activated Nanothermolysis Of Cells” filed Jul. 23, 2007;
U.S. Pat. No. 9,289,191, titled “System and method for Acquiring Optoacoustic Data and Producing Parametric Maps Thereof”, and filed Jun. 13, 2012;
U.S. Pat. No. 9,517,055, titled “System And Method For Acquiring Optoacoustic Data And Producing Parametric Maps Using Subband Acoustic Compensation” filed Nov. 25, 2013;
U.S. Pat. No. 9,724,072, titled “System And Method For Mixed Modality Acoustic Sampling” filed Dec. 13, 2013;
U.S. Pat. No. 9,456,805, titled “System And Method For Acquiring Optoacoustic Data And Producing Parametric Maps Using Interframe Persistent Artifact Removal” filed Dec. 19, 2013;
U.S. Publication 2016/0199037, titled “System And Method For Acquiring Optoacoustic Data And Producing Parametric Maps thereof” filed Mar. 22, 2016;
U.S. Publication 2017/0035388, titled “System And Method For Mixed Modality Acoustic Sampling” filed Oct. 18, 2016;
U.S. Pat. No. 9,792,686, titled “System And Method For Acquiring Optoacoustic Data And Producing Parametric Maps Using Subband Acoustic Compensation” filed Nov. 17, 2016;
U.S. Publication 2017/0296151, titled “System And Method For Mixed Modality Acoustic Sampling” filed Jun. 30, 2017;
U.S. Publication 2013/0109950, titled “Handheld Optoacoustic Probe” filed Nov. 2, 2011;
U.S. Publication 2016/0296121, titled “Handheld Optoacoustic Probe” filed May 2, 2016;
U.S. Pat. No. 8,686,335, titled “System And Method For Adjusting The Light Output Of An Optoacoustic Imaging System” filed Dec. 31, 2011;
U.S. Pat. No. 9,528,936, titled “System And Method For Adjusting The Light Output Of An Optoacoustic Imaging System” filed Mar. 31, 2014;
U.S. Publication 2017/0108429, titled “System And Method For Adjusting The Light Output Of An Optoacoustic Imaging System” filed Dec. 27, 2016;
U.S. Pat. No. 9,330,452, titled “Statistical Mapping In An Optoacoustic Imaging System” filed Mar. 11, 2013;
U.S. Pat. No. 9,836,838, titled “Statistical Mapping In An Optoacoustic Imaging System” filed May 3, 2016;
U.S. Publication 2018/0061050, titled “Statistical Mapping In An Optoacoustic Imaging System” filed Nov. 6, 2017;
U.S. Pat. No. 9,610,043, titled “System And Method For Producing Parametric Maps Of Optoacoustic Data” filed Jun. 13, 2012;
U.S. Publication 2017/0100040, titled “System And Method For Producing Parametric Maps Of Optoacoustic Data” filed Dec. 21, 2016;
U.S. Publication 2013/0338501, titled “System And Method For Storing Data Associated With The Operation Of A Dual Modality Optoacoustic/Ultrasound System” filed Jun. 13, 2012;
U.S. Publication 2013/0338475, titled “Optoacoustic Imaging System With Fiber Optic Cable” filed Jun. 13, 2012;
U.S. Publication 2014/0194723, titled “Multi-Layer Coating For Optoacoustic Probe” filed Jan. 13, 2014;
U.S. Publication 2017/0150890, titled “Optoacoustic Probe With Multi-Layer Coating” filed Jan. 31, 2017;
U.S. Pat. No. 9,615,750, titled “Methods And Compositions For Carrier Agents And Clearing Agents Used In Optoacoustic Imaging Systems” filed Jun. 14, 2012;
U.S. Publication 2013/0116538, titled “Optoacoustic Imaging Systems And Methods With Enhanced Safety” filed Oct. 19, 2012;
U.S. Publication 2015/0297090, titled “Optoacoustic Imaging Systems And Methods With Enhanced Safety” filed Jan. 23, 2015;
U.S. Publication 2013/0289381, titled “Dual Modality Imaging System For Coregistered Functional And Anatomical Mapping” filed Nov. 2, 2012;
U.S. Pat. No. 9,757,092, titled “Method For Dual Modality Optoacoustic Imaging” filed Nov. 2, 2012;
U.S. Publication 2014/0039293, titled “Optoacoustic Imaging System Having Handheld Probe Utilizing Optically Reflective Material” filed Jan. 22, 2013;
U.S. Publication 2017/0014101, titled “Dual Modality Imaging System For Coregistered Functional And Anatomical Mapping” filed Sep. 27, 2016;
U.S. Publication 2013/0303875, titled “System And Method For Dynamically Varying The Angle Of Light Transmission In An Optoacoustic Imaging System” filed Nov. 2, 2012;
U.S. Pat. No. 9,445,785, titled “System And Method For Normalizing Range In An Optoacoustic Imaging System” filed Dec. 21, 2012;
U.S. Pat. No. 9,282,899, titled “System And Method For Detecting Anomalous Channel In An Optoacoustic Imaging System” filed Dec. 21, 2012;
U.S. Publication 2014/0005544, titled “System And Method For Providing Selective Channel Sensitivity In An Optoacoustic Imaging System” filed Dec. 21, 2012;
U.S. Publication 2016/0317034, titled “System And Method For Providing Selective Channel Sensitivity In An Optoacoustic Imaging System” filed Jul. 11, 2016;
U.S. Pat. No. 9,445,786, titled “Interframe Energy Normalization In An Optoacoustic Imaging System” filed Jan. 22, 2013;
U.S. Publication 2017/0000354, titled “Interframe Energy Normalization In An Optoacoustic Imaging System” filed Sep. 19, 2016;
U.S. Publication 2014/0206978, titled “Probe With Optoacoustic Isolator” filed Jan. 22, 2013;
U.S. Pat. No. 9,743,839, titled “Playback Mode In An Optoacoustic Imaging System” filed Mar. 15, 2013;
U.S. Publication 2017/0332916, titled “Playback Mode In An Optoacoustic Imaging System” filed Jul. 27, 2017;
U.S. Pat. No. 9,398,893, titled “System And Method For Diagnostic Vector Classification Support” filed Mar. 11, 2014;
U.S. Pat. No. 10,026,170, titled “System And Method For Diagnostic Vector Classification Support” filed Jul. 19, 2016;
U.S. Application number Ser. No. 16/022,138, titled “System And Method For Diagnostic Vector Classification Support” filed Jun. 28, 2018;
U.S. Pat. No. 9,730,587, titled “Diagnostic Simulator” filed Mar. 15, 2013;
U.S. Publication 2017/0332915, titled “Diagnostic Simulator” filed Jul. 27, 2017;
U.S. Pat. No. 8,823,928, titled “Light Output Calibration In An Optoacoustic System” filed Mar. 15, 2013;
U.S. Pat. No. 9,163,980, titled “Light Output Calibration In An Optoacoustic System” filed Jul. 11, 2014;
U.S. Pat. No. 9,814,394, titled “Noise Suppression In An Optoacoustic System” filed Mar. 15, 2013;
U.S. Publication 2018/0078144, titled “Noise Suppression In An Optoacoustic System” filed Nov. 13, 2017;
U.S. Pat. No. 9,733,119, titled “Optoacoustic Component Utilization Tracking” filed Mar. 15, 2013;
U.S. Publication 2017/0322071, titled “Optoacoustic Component Utilization Tracking” filed Jul. 27, 2017;
U.S. Publication 2015/0101411, titled “Systems And Methods For Component Separation In Medical Imaging” filed Oct. 13, 2014;
U.S. Publication 2015/0305628, titled “Probe Adapted To Control Blood Flow Through Vessels During Imaging And Method Of Use Of Same” filed Feb. 27, 2015;
U.S. Publication 2016/0187481, titled “Opto-Acoustic Imaging System With Detection Of Relative Orientation Of Light Source And Acoustic Receiver Using Acoustic Waves” filed Oct. 30, 2015.

Provided is an optoacoustic probe with a laser light assembly having a redundant sensor to determine whether a laser light source is operating in a safe voltage range. In one example, in addition to providing a primary sensor that measures energy output of a laser that is used to provide feedback that controls the output voltage of the laser, an additional, or secondary sensor can be provided to verify the primary sensor is operating accurately. If a ratio between the primary sensor and secondary sensor exceeds a threshold value, alerts can be provided to a user, or alternatively, the probe can be automatically shut down. The redundant sensor results in an additional check on the primary sensor in addition to a system that determines the confidence level of the reading of the primary sensor. Alternatively, the redundant secondary sensor can replace the need for the confidence level system to greatly reduce costs of the optoacoustic probe.

The secondary sensor can also be used to verify the reliability of the reading produced by the primary sensor and the overall health of the laser light source. In one example, to accomplish, the expected energy output based on a command position of an attenuator of the laser light source can be compared to either the energy output detected by the primary sensor or the redundant secondary sensor. In one example the energy output detected by the primary sensor can be compared to the energy output detected by the secondary sensor to provide a ratio. This comparison and ratio can identify whether an unexpected change in the measurement system has occurred.

In one example, the primary sensor measures the energy output of a laser light path emitting from a resonator while the secondary sensor measures the energy output of the laser light path prior to entering the resonator. To address effects of potential noise or transient light associated with the resonator, a slope/offset fit of the primary sensor to an external reference meter can be provided. Once calibrated, if the ratio of the energy measured by the primary sensor compared to the energy measured by the secondary sensor exceeds a threshold an indicator is provided that a component within the resonator malfunctioning or in need of maintenance. In one example, the ratio detected between the primary sensor and the secondary sensor can be used to determine that service of the laser light system is required.

In another example, controlling the commanded light output attenuator position and using a primary sensor to monitor the laser light source energy output can allow determinations of the attenuator position which produces the maximum light energy output. In an example, monitoring subsequent laser light source output, the attenuator can be commanded to attenuator positions to maximize light energy output. In another example, the commanded laser light source output energy transmission can be determined accordingly.

In another example, the secondary sensor can be utilized to determine a laser light source output energy at the maximum light output energy attenuator position. In another example a ratio between the light energy output detected by the primary sensor and the light energy output detected by a secondary sensor can be determined and utilized in determining the health of the laser system. At any commanded light output attenuator position, the associated primary sensor energy output reading and secondary sensor energy output reading can be used in combination to determine the actual light output energy transmission in addition to the health of the laser system. In an example, by comparing the commanded light output energy transmission to the actual light output energy transmission obtained from a primary or secondary sensor, a set of program instructions can be executed on a microprocessor to determine if the energy transmission varies significantly from the expected value, and if so an alert can be produced or optionally the laser light source can be shut down.

In another example embodiment, a secondary light sensor can be used at the distal end of an optoacoustic probe to ensure proper functionality of the laser system from the laser light source to the distal end of the optoacoustic probe. In one example, the light output from the distal end of the optoacoustic probe may be coupled to a calibrated reference energy meter. A microcontroller and one or more processors containing memory and program instructions may be configured to read the output of the calibrated reference energy meter, and the signals detected by the secondary sensor at the distal end of the optoacoustic probe. By controlling the light output attenuator position, a calibration fit can be established relating the output of the first energy output to the reference energy meter. The calibrated first energy output can be monitored and combined with and varying the light output attenuator to maintain the light output energy at a given setpoint. By using the calibrated first energy output and applying the calculated first light output transmission a calibrated second energy output can be determined. This quantity can be monitored to determine the total energy of the light in the laser system.

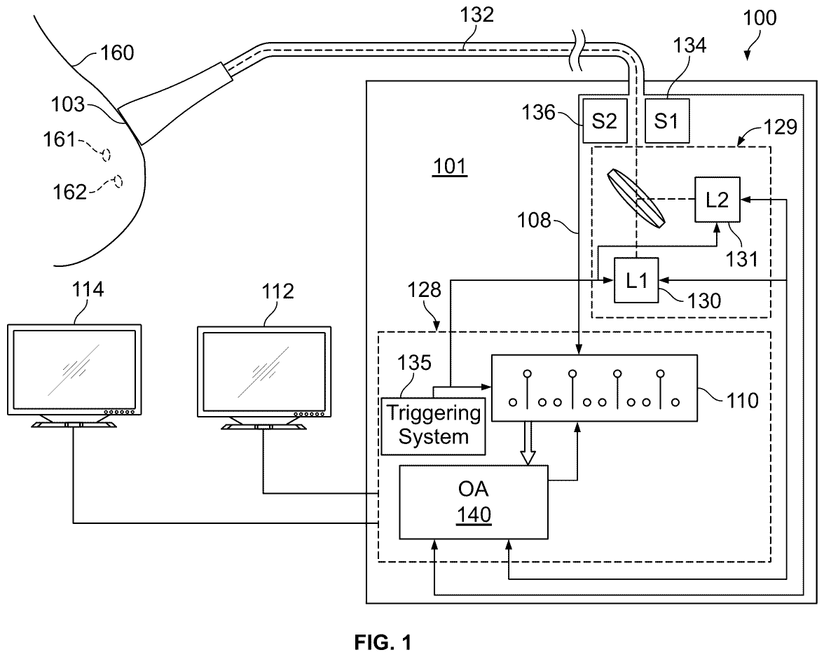

Turning to FIG. 1 device 100 provides an optoacoustic probe system. In an embodiment, the optoacoustic probe system 100 includes a probe 102 connected via a light path 132 and an electrical path 108 to a system chassis 101. Within the system chassis 101 is housed a light subsystem 129 and a computing subsystem 128. The computing subsystem 128 includes one or more computing components for optoacoustic control and analysis; these components may be separate, or integrated. In an embodiment, the computing subsystem comprises a relay system 110, a triggering system 135, an optoacoustic processing and overlay system 140.

In an embodiment, the light subsystem 129 is capable of producing pulses of light of at least two different wavelengths. In an embodiment, the light subsystem 129 includes two separate light sources 130, 131. In one example at least one (or both) of the light sources 130, 131 are laser light sources. In an embodiment the light sources 130, 131 area Nd: YAG laser and an Alexandrite laser. The output of the primary light sources 130, 131 of the light subsystem 129 is delivered to the probe 102 via the light path 132.

The light subsystem 129 in one embodiment can include a first energy output sensor 134 and a second energy output sensor 136. In examples the first energy output sensor 134 and second energy output sensor 136 can be pyroelectric sensors, photodiode sensors, a combination of a pyroelectric sensor and a photodiode sensor, or the like. In one example the first energy output sensor 134 and second energy output sensor 136 can be positioned adjacent to a light source 130 or 131 within the chassis 101. In another example the first energy output sensor 134 and the second energy output sensor 136 can be positioned adjacent to a window 103 of the optoacoustic probe 102 where the light path 132 exits the probe 102. In yet another example a first energy output sensor 134 can be positioned within the chassis 101 while the second energy output sensor 136 is positioned within the probe 102 or one the probe 102. In each example the first energy output sensor and second energy output sensor are able to detect or be used to determine the energy output of one or more light sources 130, 131.

The computing subsystem 128 can receive signals or readings from each of the first energy output sensor 134 and the second energy output sensor 136 to identify or determine the energy output of one or more of the light sources 130, 131. In one example the computing subsystem can include a subsystem for determining the confidence level of at least one, if not both of the first energy output sensor 134 and the second energy output sensor 136. In an example, the computing subsystem can be configured to use the energy output identified or determined to dynamically adjust the voltage provided for at least one light source 130, 131 to keep the energy output of the light source 130, 131 between 80%-120% of a target energy output. In an example the computing subsystem determines the expected energy output based on the voltage provided and compares the detected energy output and based on this comparison adjusts the voltage to ensure the energy output remains within the 80%-120% range. So, if the voltage is set to achieve a 100% energy output and one or more of the first energy output sensor 134 and/or second energy output sensor 136 are used to determine the detected energy output is only 70% of the expected energy output, the computing subsystem 128 may cause the output voltage to increase the detected energy output.

In one example, the computing subsystem 128 can also be configured to compare the energy output detected by the first energy output sensor 134 and the second energy output sensor 136. Based on this ratio from the comparison a 1:1 ratio would be expected. In one example, if the ratio exceeds a threshold amount, such as 25% of the 1:1 ratio, the computing subsystem can provide an alert to a user. In one example the alert can be provided on one or more displays 112, 114. The one or more displays 112, 114 may be touch screen displays that are provided for displaying images and all or portions of the optoacoustic probe system 100 user interface. By communicating the alert on a display 112, 114 where the image is located, a clinician using the probe 102 can immediately be alerted that performance or safety may be compromised, or that maintenance of the probe is required.

In another example, once reaching the threshold, the computing subsystem 129 may automatically actuate the triggering system to turn off the one or more light sources 130, 131. In another example, a first threshold may result in an alert or communication being provided, while a second different threshold may be provided that results in the actuation of the trigging system to turn off the one or more light sources 130, 131. In yet another example, once a threshold is reached a timer can be started and if the threshold remains exceeded for a determined period of time an alert is communicated, or the triggering system turns off one or more of the light sources 130, 131. In yet another example, even if the determined period of time is not reached, if the threshold is exceeded for a determined amount of time during a period the alert can be communicated or triggering system can actuate to turn off one or more light sources 130, 131. In one example the determined amount of time can be five seconds, and the period can be thirty seconds. In another example the determined amount of time can be ten seconds, and the period can be two minutes.

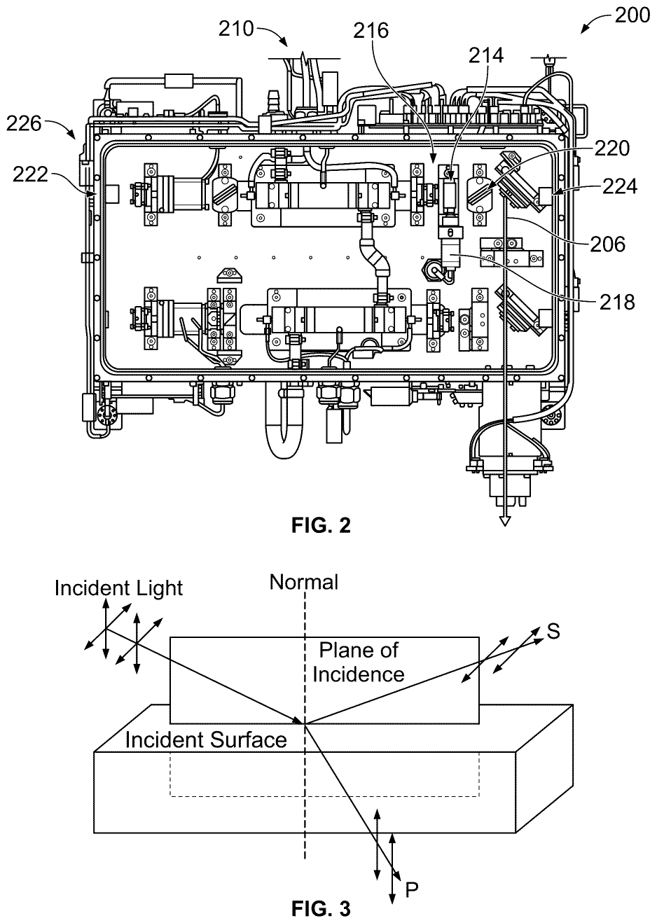

FIG. 2, illustrates and example partial laser light assembly 200 that receives a laser light path output 206 from a first laser light source or a second laser light source for conditioning. In one example the first laser light source can be an Alexandrite laser while the second laser light source can be a Nd: YAG laser. When using an Alexandrite laser, ensuring a consistent laser path output 206 is desired for regulation and safety purposes. The laser light path can include an attenuator 210 that conditions or modifies the laser light along the laser light path using numerous components. In one example the attenuator 210 can include a half waveplate 214 attached to a rotational stage 216 that can be controlled and operated by an attenuator motor 218. The attenuator 210 can also include a polarizer 220 place in a fixed position after the half waveplate.

In one example the half waveplate rotation controls the ratio of P to S light polarization to the plane of incidence as illustrated in FIG. 3. In another example the polarizer 220 splits the light into P and S components, reflecting one while transmitting the other. As the attenuator 210 is rotated 2π radians (e.g., 360 degrees), the output cycles from minimum to maximum transmission four (4) times.

With reference back to FIG. 2, in one example, a primary sensor 222 can be positioned after the attenuator 210 along the laser path, while a secondary sensor 224 can be positioned between the laser light sources and the attenuator 210. By being placed prior to the laser light entering the attenuator 210, if the energy output determined using the primary sensor 222 is different than the energy output determined using the secondary sensor 224, a malfunctioning component within the attenuator 210 can be identified. In one example the primary sensor 222 can be a first photodiode and the secondary sensor 224 a second, or redundant photodiode.

In one example, the light hitting the primary sensor 222 can be converted by a microcontroller using an analog to digital converter ADC circuit and is represented discrete values in a fixed range such as 0 to 1023 for a 10-bit ADC or 0 to 16383 for a 14-bit. To prevent saturation, the photodiode circuit can be configured such that a threshold amount of light produced by the first laser light source is closer to the middle of the range instead of the threshold. Saturation can be the condition where a value cannot be reliability measured because the amount of signal exceeds the capability of the measurement system.

In an example the laser light assembly 200 can optionally include a polarizer filter 226 in front of the primary sensor 222 that can be adjusted to control the amount of light hitting the primary sensor 222. In another example, when no light is being produced, a non-zero value, or offset, can be determined by the microcontroller. In an example the offset can be provided when the half waveplate 214 and/or polarizer 220 are offset or damaged and allows light to leak through. In another example minor fluctuations in the reading can be due to the presence of electrical noise and/or variations in the primary laser light output and are reflected in the primary sensor 222. These variations can normally be small compared to the total output and are usually quantified as the standard deviation of the laser output. The total equation is below and is a visualization of the expected waveform:

Photodiode_{counts}=(Photodiodeₘₐₓ−Photodiode_{offset} noise)*cos2(2X)+Photodiode_{offset}+noise

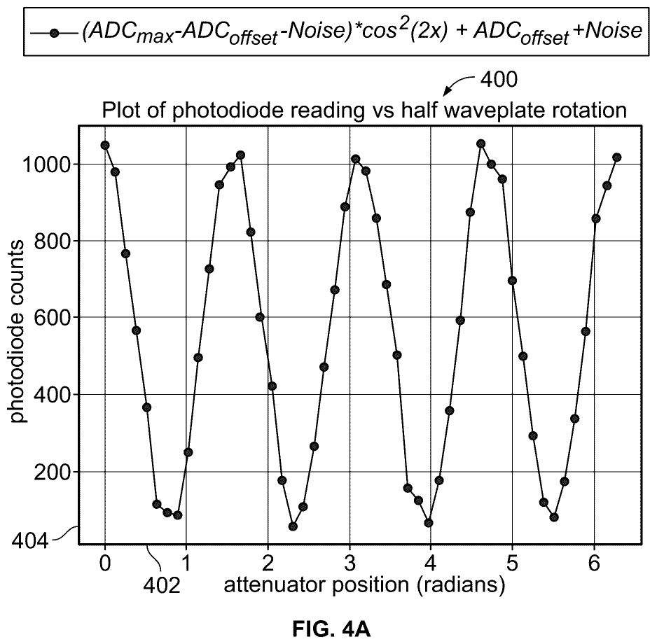

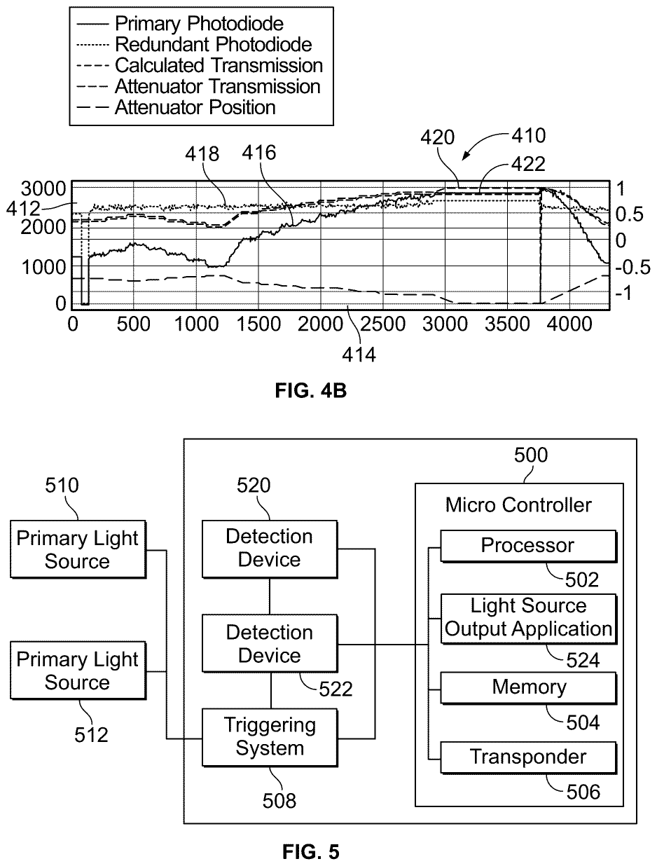

FIG. 4A illustrates a simulated plot 400 of sample readings from the primary sensor 222 by the ADC over the full attenuator range with the inclusion of offset and noise is below. (assuming that a maximum range of values of 1023 is present). The graph presents the attenuator position 402 as measured in radians compared to the readings 404 of a primary sensor as measured in counts.

FIG. 4B illustrates a graph 410 of energy 412 over time 414 for a laser light system. Illustrated are the primary sensor energy output 416 and the secondary sensor energy output 418. In one example, over time the energy produced by the first laser light source increases. In another example the energy of the first laser light source increases based on a cold start. A cold start is when a laser is actuated immediately or in close proximity to the time to the laser system being turned on after an extended period of non-use. Alternatively after the laser system is turned on, a period of time is provided for the laser light source to warm up before use. While in this instance (e.g., when a period of time is provided for warming up the first laser light source) increasing energy levels can be realized, such increasing energy levels are not as common as compared to the cold start operation.

As illustrated, the redundant secondary sensor represents the total energy in the resonator, whereas the primary sensor represents the energy exiting the laser, or attenuated laser energy In one example, when the transmission=100% the redundant photodiode reading can be a scaled representation of the primary sensor. For example:

A0*Eredundant=Eprimary

Where A0 represents the scaling factor.

In other instances, where the laser output is attenuated. The relationship is as follows:

A0*Eredundant*cos2(2X)=(Eprimary−Eoffset)

Where the transmission is T=cos2(2X) such that

T=1 A0*Eprimary−EoffsetEredundant

In one example A0 can be determined by identifying the maximum energy position of the attenuator, and the offset can be determined by collecting a reading without the laser firing, or at the minimum energy position of the attenuator.

As such, FIG. 4B illustrates the primary sensor energy output readings 416 compared to the secondary sensor energy output readings 418 during a calibration process. Based on these readings and the above calculations, the input for the first laser light source can be controlled.

When the transmission, or input for the first laser light source is known and the first and second energy output readings 416 and 418 are known, a determination can be made related to the amount of energy in the laser resonator. The amount of energy in the laser resonator can be calculated as follows:

### If

Eredundant*T=Eprimary

### Then

Eredundant=Eprimary/T

Where Eprimary is the first energy output of the primary sensor and Eredundant is the second energy output of the secondary sensor.

By comparing the attenuator transmission 420 compared to the calculated transmission 422 using the above calculations, damaged components within the laser light assembly can be identified. For example, a sudden reduction or rate of change of the resonator energy over time can indicate a faulty component within the resonator. Resonator components that may be faulty can include a high reflector mirror, pockels cell, polarizer rod, flashlamp, or the like. Alternatively, the calculations can be used to identify half waveplate damage. When the laser light emitted by the first laser light source emits over a burn or damaged spot (as a result of an optical coating being burned, or otherwise) in the half waveplate, the light output is reduced. In another example a burn or other damage to the half waveplate could cause light output to increase. In either example, a sudden change in the transmission can be indicative of the damage of the half waveplate. As another example, damage to the polarizer also would result in a sudden light output increase or decrease for similar reasons as the half waveplate. In the example, again, sudden changes in the transmission can detect the faulty component.

With reference back to FIG. 2, in yet another example, secondary sensor 224 may be disposed at the distal end of the optoacoustic probe (FIG. 1). In one example the secondary sensor 224 can be disposed adjacent to a window of the optoacoustic probe. In another example the auxiliary energy sensor can be disposed external to the optoacoustic probe and on the optoacoustic probe housing. In yet another example, a first secondary sensor can be disposed in the chassis adjacent the primary laser light source and a second secondary sensor can be placed in or on the optoacoustic probe. When a secondary sensor 224 is provided at, or associated with the optoacoustic probe, the relationship between the primary sensor 222 and the secondary sensor 224 can be used to identify faulty or malfunctioning optical fiber that carry the light along the light path from the first laser light source to the optoacoustic probe. By using one or more secondary sensors, the energy output sensed by the secondary sensor compared to that sensed by the primary sensor can be used to identify malfunctioning components within the optoacoustic probe system.

FIG. 5 illustrates a block diagram of a computing subsystem, or microcontroller 500 for an optoacoustic probe. In one example the controller and optoacoustic probe are the computing subsystem and optoacoustic probe of FIG. 1. The microcontroller 500 can include one or more processors 502 for making determinations, a memory 504 for storing instructions and historical data, including look up tables, and a transceiver 506 or transponder for receiving and transmitting communication signals to and from the probe 502. In one example the transceiver 506 can be a wireless transceiver that can communicate over a network.

The microcontroller 500 can be coupled to a triggering system 508 for a first laser light source 510 and a second laser light source 512. The triggering system 508 can be configured to independently actuate the first laser light source 510 and second laser light source 512. In one example the triggering system can turn off or stop actuating the first laser light source 510 while continuing to actuate the second laser light source. Alternatively, the triggering system 508 can simultaneously stop actuation of both the first laser light source 510 and the second laser light source 512. In one example the triggering system 508 can be configured to control and vary the amount of voltage input to each of the first laser light source 510 and second laser light source 512. In an example, as the voltage input to an individual laser light source 510, 512 increases, the energy output by that individual light source increases. In one example the increase may be a proportional increase. In one embodiment the triggering system 508 can be configured to increase or decrease the voltage for the first laser light source 510 while keeping the voltage input to the second laser light source 512 the same. In one example the triggering system 508 can be configured to increase or decrease the voltage input for the first laser light source 510 while also increasing or decreasing the voltage input for the second laser light source. In one example the voltage increase or decrease related to a first laser light source 510 can be proportional to the voltage increase or decrease related to the second laser light source 512. Alternatively, the voltage increase or decrease related to the first laser light source 510 source can be disproportional to the increase or decrease related to the second laser light source 512.

In an example the first and second laser light sources can emit visible light. In another example the first and second laser light sources can emit infrared light. In another example the first and second laser light sources can emit ultraviolet light. In an example the first laser light source 510 can emit light in a first range of wavelengths while the second laser light source 512 can emit light in a second range of wavelengths that differs from the first range of wavelengths. In an embodiment the laser light sources 510, 512 can be a Nd: YAG laser and an Alexandrite laser.

With reference back to the controller 500, the controller can also include at least one interface. In one example the interface can be a display that can be utilized to display an image by using the optoacoustic probe. The interface can include an input and an output. In one example the interface can be a touch screen that functions as both the input and output. Alternatively, the input can be a keyboard, mouse, microphone, or the like while the output can be a screen, speaker, light indicator, or the like.

The microcontroller 500 can also include a primary sensor 520 and at least one secondary sensor 522. The primary sensor 520 and secondary sensor 522 can be pyroelectric sensors, photodiode sensors, a combination of a pyroelectric sensor and a photodiode sensor, or the like. In one example the primary sensor 520 can be positioned within a chassis of an optoacoustic probe system after an attenuator of the laser path. In another example the secondary sensor 522 can be posited in front of the attenuator within the chassis. In another example the secondary sensor can be placed within or on an optoacoustic probe. In yet another example the microcontroller can operate plural secondary sensors, including as least one within the chassis and at least another in or on the optoacoustic probe.

In an embodiment the primary sensor 520 and secondary sensor 522 can detect characteristics of interest related to the energy output by the first laser light source 510 and second laser light source 512. In one example the characteristics of interest can be used to determine, calculate, estimate, derive, or the like the energy output of the first laser light source 510 or second laser light source 512. The primary sensor 520 and secondary sensor 522 can be located anywhere within the optoacoustic system to obtain or detect the energy, characteristic of interest, etc.

The microcontroller 500 can also include a light source output application 524 that is configured to obtain the readings, signals, etc. from the primary sensor 520 and the secondary sensor 522 and determine, calculate, identify, etc. the energy output by each of first and second laser light sources 510, 512. In addition, the light source output application 524 can be configured to determine the voltage being applied by the triggering system to each individual laser light source 510, 512 and the expected energy output for each light source. Based on a comparison, calculation, lookup table, mathematical model, or the like the light source output application 524 can make a determination and vary the voltage applied to either laser light source 510, 512 to ensure the energy output from each laser light source 510, 512 is within an expected range. In one example the expected range is between 80%-120% of the expected energy output. In one example the light source output application 524 can be configured to vary the voltage to have the detected energy output to be 100% of the expected energy output.

The light source output application 524 can also be configured to compare the first energy output determined using the primary sensor 520 and compare it to the second energy output determined using the secondary sensor 522. Based on the comparison a ratio can be provided and a threshold applied. In one example the threshold can be a percentage such as within 25% of a one-to-one ratio. In another example the threshold can include a first threshold and a second threshold. In one example the first threshold can be within 10% of a one-to-one ratio while the second threshold can be within 30% of a one-to-one ratio.

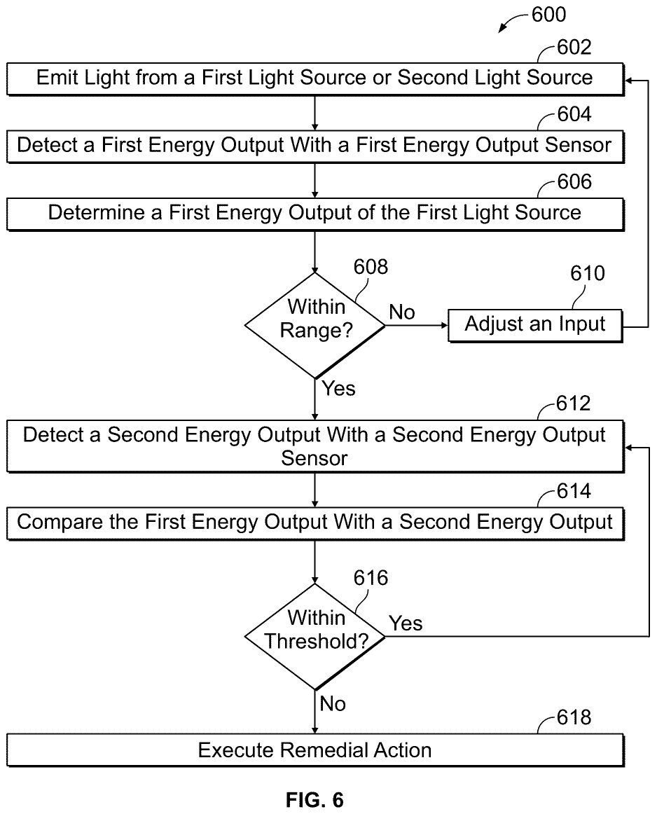

FIG. 6 illustrates a method 600 of controlling light sources of an optoacoustic probe. In example embodiments the optoacoustic systems, optoacoustic probes, controllers, etc. previously described in FIGS. 1-5 are used to complete one or more steps of the method 600.

At 602, light is emitted from at least one of a first laser light source or a second laser light source of an optoacoustic probe to obtain an optoacoustic image. In one example at least one of the first laser light source and/or the second laser light source can be an Alexandrite laser.

At 604, a primary sensor detects the amount of energy being output by the light of the first laser light source and/or the second light source. In example embodiments the primary sensor can be a pyroelectric sensor, a photodiode sensor, or the like. Alternatively, the primary sensor can be a sensor that functions to detect a characteristic of interest that can be used to determine the energy output of the light emitted by one or more of the first laser light source and/or the second laser light source.

At 606, one or more processors determine the amount of energy being output by one of the first laser light source or second laser light source based on the reading related to the primary sensor. At 608, the one or more processors determine whether the energy being output by the first laser light source, or second laser light source are within a determined range of a ratio. For example, the expected energy output could be 100 millijoules (mJ) per pulse such that when the detected energy output is 100 mJ per pulse the actual ratio would be 100%. Whereas if the expected energy output was 100 mJ per pulse and the detected energy output is 110 mJ per pulse, the actual ratio would be 110%. In one example the determined range can be between 80%-120%. In another example the determined range can be between 75%-125%. In yet another example the determined range can be between 90%-110%.

At 608, if the ratio is not within the determined range, then at 610 the one or more processors dynamically adjust an input of the first light source and/or second light source to vary the energy output. For example, if the determined range is between 80%-120% and the 130 mJ per pulse is detected when 100 mJ per pulse is expected to be detected, a ratio of 130% is determined. In response the one or more processors reduce an input, such as voltage, to decrease the energy output by the first light source or second light source. In one example the one or more processors determine the amount of variance in the input based on the variance between the expected ratio and the actual ratio. In one example the greater the variance, the greater the variance in the input.

After the input is varied at 610, this feedback process continues until the energy output detected is within the determined range. When the determinations are made, the initial expected energy output remains the same even though the input voltage is varied. Thus, in the example above, 100 mJ remains the expected energy output such that the system calibrates the voltage to achieve that output.

If at 608 the actual ratio is within range, at 612 the energy output is detected by a second energy output sensor. The second energy output sensor can be any of the energy output sensors that the first energy output sensor can be. The second energy output sensor is a redundant sensor that can be used to verify that the first energy output sensor is providing consistent readings.

At 614, the first energy output determined using the primary sensor is compared to the second energy output determined using the secondary sensor. Because both the primary sensor and secondary sensor are measuring the same light, the two determinations are expected to be identical, or have a one-to-one ratio.

At 616, a determination is made whether the comparison between the first energy output determined and the second energy output determined exceeds a determined threshold. In one example the determined threshold can be a percentage such as 10%. In one example the determined threshold can be a number of mJ. For example, if the first energy output determined is 100 mJ per pulse and the second energy output determined is 90 mJ per pulse and the determined threshold is 5% the threshold exceeded. Similarly, if the determined threshold is 5 mJ, again the threshold is exceeded. Alternatively, the threshold may have to occur for a period of time. So, if the 5% or 5 mJ threshold is exceeded for five consecutive second, or ten seconds in a minute, then the threshold is considered exceeded. If the threshold is not exceeded, the method continues until the threshold is exceeded or the imaging ends.

At 618, if the threshold is exceeded the one or more processors execute a remedial action. In one example the remedial action is communicating a warning or alert on a display that the faulty readings may be occurring. In another example, the remedial action can be turning off, or preventing actuation of the first and/or second laser light source. In another example, an alert may be communicated based on a first threshold and the first and/or second laser light source turned off or prevented from actuating based on a second threshold. For example, a first threshold can be 5% or 5 mJ while a second threshold can be 10% or 10 mJ. So, if a difference of 6 mJ or 6% is detected, a first remedial action, that alerts a user of potential inaccurate results on a display can occur. Alternatively, if the difference is 12 mJ or 12%, the remedial action can be shutting down the probe, or preventing actuation of the first light source and/or second light source.

By using the redundant energy output sensor a method of ensuring a laser light source is properly functioning is provided. The additional method can be used simultaneously with curve fitting or other methodologies to verify the functioning of laser light sources. The system and method provided enhance safety, accuracy, and functionality accordingly.

The present system and methods are described above with reference to block diagrams and operational illustrations of methods and devices comprising an optoacoustic probe. It is understood that each block of the block diagrams or operational illustrations, and combinations of blocks in the block diagrams or operational illustrations, may be implemented by means of analog or digital hardware and computer program instructions. These computer program instructions may be provided to a processor of a general purpose computer, special purpose computer, ASIC, FPGA or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g., 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about,” unless the context clearly dictates otherwise. Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Those skilled in the art will recognize that the methods and systems of the present disclosure may be implemented in many manners and as such are not to be limited by the foregoing example embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto. Thus, while the invention has been particularly shown and described with reference to a preferred embodiment thereof, it will be understood by those skilled in the art that various changes in form and details may be made therein without departing from the spirit and scope of the invention.

What is claimed is:

1. An optoacoustic probe system for optoacoustic imaging of a volume comprising:
an optoacoustic probe having a distal end operable to contact the volume and a proximal end, the optoacoustic probe;
at least one laser light source outside a probe housing configured to generate light that is transmitted along a light path to generate optoacoustic return signals when the light reacts with the volume;
a primary sensor configured to detect signals from the light generated by the at least one laser light source;
a secondary sensor configured to detect the signals from the light generated by the at least one laser light source;
a microcontroller including one or more processors, and a memory coupled to the one or more processors, wherein the memory stores program instructions, wherein the program instructions are executable by the one or more processors to:
detect signals from the light with the primary sensor;
determine a first energy output based on the signals detected by the primary sensor;
detect the signals with the secondary sensor;
determine a second energy output based on the signals detected by the secondary sensor;
compare the first energy output to the second energy output; and
provide an alert or turning off the at least one laser light source based on comparing the first energy output to the second energy output.

2. The optoacoustic probe system of claim 1, wherein the one or more processors are further configured to:
determine whether the first energy output is within a range of the second energy output based on a comparison; and
provide the alert or turning off the at least one laser light source in response to a determination that a comparison between the first energy output to the second energy output falls outside of the range.

3. The optoacoustic probe system of claim 1, the one or more processors further configured to vary an input to the at least one laser light source by determining whether the first energy output is within an expected power output range related to the at least one laser light source and varying the input in response to the first energy output falling outside of the expected power output range.

4. The optoacoustic probe system of claim 3, wherein the input is a voltage input to the at least one light source.

5. The optoacoustic probe system of claim 1, wherein the at least one light source is a Alexandrite laser.

6. The optoacoustic probe system of claim 1, wherein the at least one laser light source is disposed within a chassis.

7. The optoacoustic probe system of claim 6, wherein the primary sensor is disposed within the chassis adjacent the at least one laser light source.

8. The optoacoustic probe system of claim 7, wherein the secondary sensor is disposed within the chassis adjacent the at least one laser light source.

9. The optoacoustic probe system of claim 1, wherein the primary sensor is at least one of a photodiode sensor or a pyroelectric sensor.

10. The optoacoustic probe system of claim 9, wherein the secondary sensor is at least one of a photodiode sensor or a pyroelectric sensor.

11. The optoacoustic probe of claim 1, wherein the one or more processors are configured to provide the alert and to provide the alert includes displaying on a display a message.

12. The optoacoustic probe of claim 11, wherein the display is configured to display an optoacoustic image.

13. An optoacoustic probe system for optoacoustic imaging of volume comprising:
an optoacoustic probe having a distal end operable to contact the volume and a proximal end, the optoacoustic probe;
a chassis having at least one laser light source configured to generate light that is transmitted along a light path between the chassis and optoacoustic probe to generate optoacoustic return signals when the light reacts with the volume;
a display disposed on the chassis and configured to display an optoacoustic image based on the optoacoustic return signals;
a primary sensor configured to detect signals from the light generated by the at least one laser light source;
a secondary sensor configured to detect the signals from the light generated by the at least one laser light source;
a microcontroller including one or more processors, and a memory coupled to the one or more processors, wherein the memory stores program instructions, wherein the program instructions are executable by the one or more processors to:
detect the signals with the primary output sensor;
determine a first energy output based on the signals detected by the primary sensor;
detect the signals with the secondary output sensor;
determine a second energy output based on the signals detected by the secondary sensor;
compare the first energy output to the second energy output; and
display an alert on the display based on comparing the first energy output to the second energy output.

14. The optoacoustic probe system of claim 13, wherein the one or more processors are further configured to determine whether a comparison of the first energy output and the second energy output is within a first range; and display the alert when the comparison is within the first range.

15. The optoacoustic probe system of claim 14, wherein the one or more processors are further configured to determine whether when comparing the first energy output and the second energy output is within a second range that is greater than the first range; and turn off the at least one laser light source when the comparison between the first energy output and the second energy output is outside the second range.

16. The optoacoustic probe system of claim 13, the one or more processors further configured to vary an input to the at least one laser light source by determining whether the first energy output is within an expected power output range related to the at least one laser light source and varying the input in response to the first energy output falling outside of the expected power output range.

17. The optoacoustic probe system of claim 16, wherein the input is a voltage input to the at least one laser light source.

18. The optoacoustic probe system of claim 13, wherein the at least one laser light source is an Alexandrite laser.

19. The optoacoustic probe system of claim 13, wherein the primary sensor is disposed within the chassis adjacent the at least one laser light source.

20. The optoacoustic probe system of claim 19, wherein the secondary sensor is disposed within the chassis adjacent the at least one laser light source.
